(19) United States
(12) Patent Application Publication
(10) Pub. No.: US 2018/0045697 A1
(43) Pub. Date: Feb. 15, 2018

(54) THERMAL DESORPTION SYSTEM AND METHOD OF ANALYZING A SUBSTRATE USING THE SAME

(71) Applicant: SAMSUNG ELECTRONICS CO., LTD., Suwon-si (KR)

(72) Inventors: Kyung-Ju SUK, Hwaseong-si (KR); Eun-Hee PARK, Yongin-si (KR); Sang-Hwan KIM, Seoul (KR); Hye-Kyoung MOON, Yongin-si (KR); Jung-Dae PARK, Yongin-si (KR); Min-Soo SUH, Hwaseong-si (KR); Kwang-Shin LIM, Yongin-si (KR)

(21) Appl. No.: 15/458,395

(22) Filed: Mar. 14, 2017

(30) Foreign Application Priority Data
Aug. 10, 2016 (KR) 10-2016-0101659

(51) Int. Cl. G01N 33/00; G01N 25/00; G01N 1/44

(52) U.S. Cl. CPC G01N 33/0011; G01N 1/44; G01N 25/00; G01N 33/0036

(57) ABSTRACT

A thermal desorption system including a chamber including a space in which a substrate is heated; a flow compartment within the chamber, the flow compartment providing a separate gas flow space within the chamber; a substrate support that supports the substrate within the flow compartment; a heater that heats the substrate within the flow compartment; and a gas pipe that introduces a carrier gas into the flow compartment and discharges the carrier gas from the flow compartment.

### CROSS-REFERENCE TO RELATED APPLICATION

Korean Patent Application No. 10-2016-0101659, filed on Aug. 10, 2016, in the Korean Intellectual Property Office, and entitled: “Thermal Desorption System and Method of Analyzing A Substrate Using the Same,” is incorporated by reference herein in its entirety.

### BACKGROUND

### 1. Field

Embodiments relate to a thermal desorption system and a method of analyzing a substrate using the same.

### 2. Description of the Related Art

During semiconductor manufacturing processes, a reaction gas may be adsorbed on a surface of a layer formed on a wafer, thereby causing a failure. A thermal desorption system may desorb the material adsorbed on the wafer surface and analyze the desorbed material.

### SUMMARY

Embodiments are directed to a thermal desorption system and a method of analyzing a substrate using the same.

The embodiments may be realized by providing a thermal desorption system including a chamber including a space in which a substrate is heated; a flow compartment within the chamber, the flow compartment providing a separate gas flow space within the chamber; a substrate support that supports the substrate within the flow compartment; a heater that heats the substrate within the flow compartment; and a gas pipe that introduces a carrier gas into the flow compartment and discharges the carrier gas from the flow compartment.

The embodiments may be realized by providing a thermal desorption system including a chamber including a lower chamber and an upper chamber, the lower chamber and the upper chamber being engaged with each other to provide a first space; a flow compartment within the chamber to provide a separate second space within the first space; a substrate support that supports the substrate within the flow compartment; a heater that heats the substrate within the flow compartment; and a gas pipe that introduces and discharges a carrier gas into and from the flow compartment.

The embodiments may be realized by providing a thermal desorption system including a flow compartment; a substrate support in the flow compartment, a substrate being supportable on the substrate support; a heater in the flow compartment, the substrate being heatable by the heater such that a material is desorbable from a surface of the substrate; a gas pipe in fluid communication with the flow compartment, a carrier gas being introducible into the flow compartment and dischargeable from the flow compartment through the gas pipe; and an analyzer that analyzes gas discharged through the gas pipe for the presence of a material desorbed from a surface of the substrate.

### BRIEF DESCRIPTION OF THE DRAWINGS

Features will be apparent to those of skill in the art by describing in detail exemplary embodiments with reference to the attached drawings in which:

FIG. 1 illustrates a schematic view of a thermal desorption system in accordance with example embodiments.

FIG. 2 illustrates a plan view of a lower chamber of the thermal desorption system in FIG. 1.

FIG. 3 illustrates a perspective view of the lower chamber in FIG. 2.

FIG. 4 illustrates a schematic view of an analyzer of the thermal desorption system in FIG. 1.

FIG. 5 illustrates a flow compartment of the thermal desorption system in FIG. 1.

FIG. 6 illustrates a cross-sectional view of a portion of the flow compartment in accordance with other example embodiments.

FIG. 7 illustrates a plan view of a thermal desorption system in accordance with example embodiments.

FIGS. 8 to 10 illustrate views of stages in a method of analyzing a substrate in accordance with example embodiments.

### DETAILED DESCRIPTION

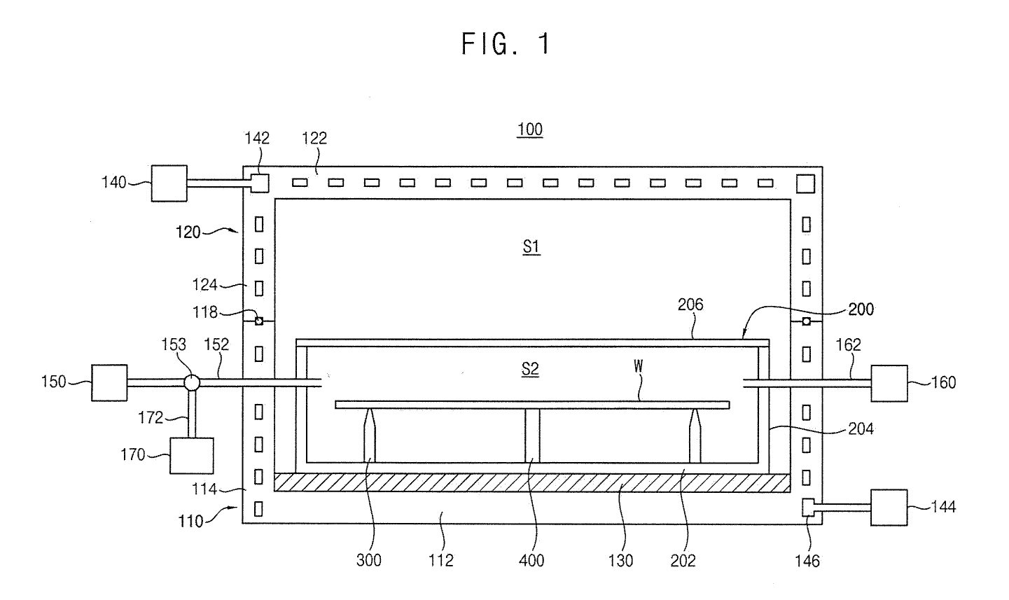

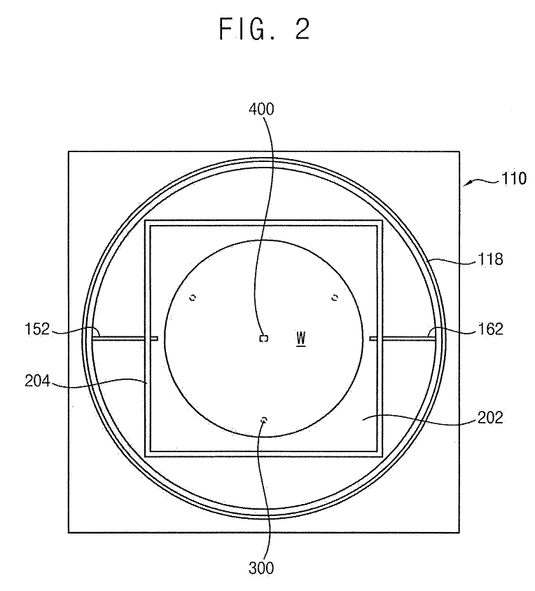

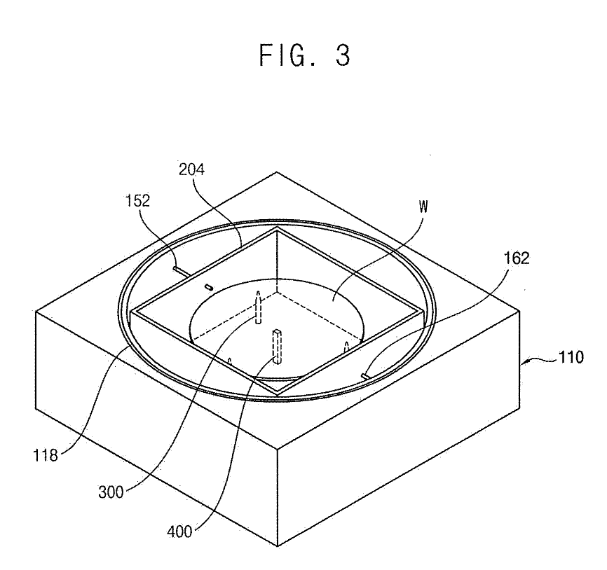

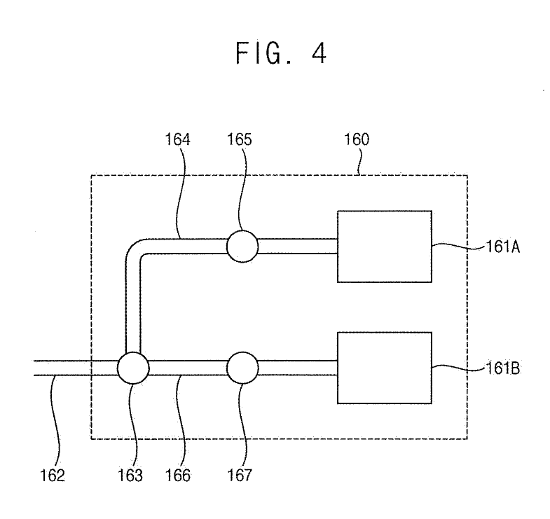

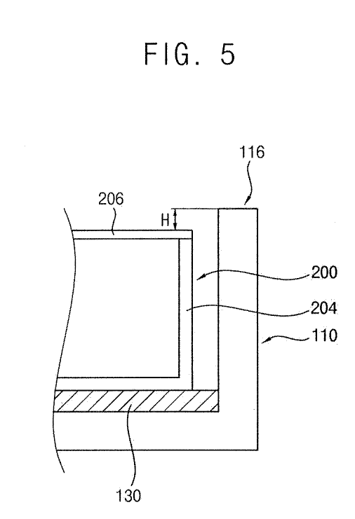

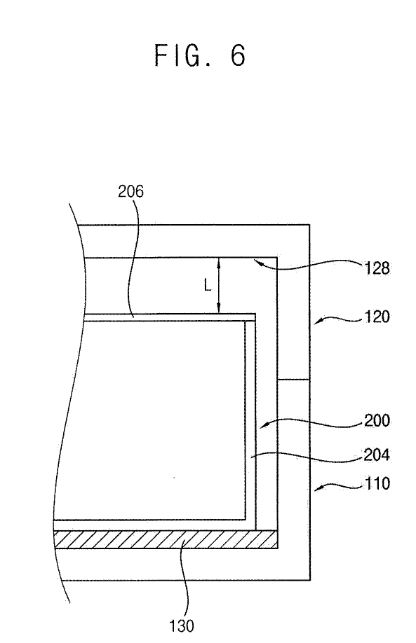

FIG. 1 illustrates a schematic view of a thermal desorption system in accordance with example embodiments. FIG. 2 illustrates a plan view of a lower chamber of the thermal desorption system in FIG. 1. FIG. 3 illustrates a perspective view of the lower chamber in FIG. 2. FIG. 4 illustrates a view of an analyzer of the thermal desorption system in FIG. 1. FIG. 5 illustrates a flow compartment of the thermal desorption system in FIG. 1. FIG. 6 illustrates a cross-sectional view of a portion of the flow compartment in accordance with other example embodiments.

Referring to FIGS. 1 to 6, a thermal desorption system 100 may include a chamber having a lower chamber 110 and an upper chamber 120 clamped together to form a first space S1 where a substrate, e.g., a wafer W, may be heated, a flow compartment 200 within the chamber to provide a second space S2 within the first space S1 and separate from the first space S1, a substrate support 130 within the flow compartment 200 to support the substrate, a heater to heat the substrate within the flow compartment 200, and a gas pipe to introduce and discharge a carrier gas into and from the flow compartment 200.

In an implementation, the thermal desorption system 100 may be a gas analyzer that heats the substrate (e.g., the wafer W) to desorb (e.g., remove) an adsorbed material from a surface of the substrate and analyze the desorbed material. For example, the thermal desorption system 100 may thermally desorb a material on a surface of the wafer W or in a layer on the wafer W (which may be formed on the wafer W by a semiconductor process such as a thin layer deposition process, an etch process, or the like), and may perform quantitative and qualitative analysis in real time.

As illustrated in FIG. 1, the lower chamber 110 may include a bottom wall 112 and a first sidewall 114 defining a first inner space. In an implementation, when viewed in a plan view, the first sidewall 114 may have a cylindrical shape. In an implementation, the first sidewall 114 may have a polygonal shape. The upper chamber 120 may include a top wall 122 and a second sidewall 124 defining a second inner space. In an implementation, when viewed in the plan view, the second sidewall 124 may have a cylindrical shape corresponding to the first sidewall 114. In an implementation, the second sidewall 124 may have a polygonal shape. A plating layer may be formed on surfaces of the lower chamber 110 and the upper chamber 120. For example, the chamber may include a metal such as aluminum (Al), and the plating layer may include a metal such as gold (Au).

The first sidewall 114 may have an upper edge 116, and the second sidewall 124 may have a lower edge corresponding to the upper edge 116. The lower chamber 110 and the upper chamber 120 may be engaged with each other to form the airtight, e.g., isolated, space S1 therebetween. A sealing member, e.g., an O-ring 118, may be provided in or on at least one of engagement surfaces of the upper edge 116 and the lower edge.

The lower chamber 110 and the upper chamber 120 may be movable relative to each other. For example, the upper chamber 120 may be supported and may be movable along a vertical rail extending in a vertical direction by a linear motor. The upper chamber 120 may move upwardly by the linear motor to open the chamber and may move downwardly to engage with the lower chamber 110 to close the chamber. In an implementation, the upper chamber 120 may move to open or close the chamber through a connection linkage connected to the lower chamber 110.

The heater 130 may be on or in the bottom wall 112 of the lower chamber 110. For example, the heater 130 may include a heating plate on the bottom wall of the lower chamber 110. In an implementation, the heater 130 may include a heater coil, a heater lamp, etc.

The heater 130 may be under the substrate to heat the substrate. For example, the heater 130 may heat the substrate to a temperature of, e.g., about 600° C. to about 900° C.

In an implementation, the thermal desorption system 100 may further include a coolant circulator to circulate a coolant through a coolant line 142, 146. The coolant circulator may include, e.g., a first coolant supplier 140 and/or a second coolant supplier 144. The first coolant supplier 140 may circulate a coolant through the first coolant line 142 in the upper chamber 120 to cool the upper chamber 120. The second coolant supplier 144 may circulate a coolant through the second coolant line 146 in the lower chamber 110 to cool the lower chamber 110.

The coolant circulator may help maintain the chamber below a room or ambient temperature (e.g., about 30° C.), and may help prevent the O-ring 118 from melting at a high temperature.

In an implementation, the flow compartment 200 may be disposed within the chamber to provide the second space S2 as a separate gas flow space within the chamber space. For example, the flow compartment 200 may be within the lower chamber 110. The flow compartment 200 may be on the heater 130 on the bottom wall of the lower chamber 110. The flow compartment 200 may be spaced apart from an inner upper surface 128 of the chamber by a predetermined distance L.

For example, the flow compartment 200 may include a lower wall 202 on the bottom wall of the lower chamber 110, a plurality of sidewalls 204 extending in a vertical direction on the lower wall 202, and an upper wall 206 on the sidewalls 204. In an implementation, the flow compartment 200 may have, e.g., a polygonal shape such as rectangle, when viewed in a plan view. In an implementation, the flow compartment 200 may have, e.g., a cylindrical shape. The upper wall 206 may be movable to selectively cover the sidewalls 204 such that the upper wall 206 selectively opens and closes the flow compartment 200. Accordingly, when the upper wall 206 opens the flow compartment 200, the wafer W may be loaded into the flow compartment 200 and then, the flow compartment 200 may be closed and a thermal desorption process may be performed within the flow compartment 200.

The upper wall 206 of the flow compartment 200 may be spaced from the upper surface 128 of the chamber by a first distance, and the sidewall 204 of the flow compartment 200 may be spaced from an inner surface of the first sidewall 114 of the lower chamber 110 by a second distance. The first distance may be greater than the second distance. In an implementation, the sidewall 204 of the flow compartment 200 may contact the inner surface of the first sidewall 114 of the lower chamber 110.

The flow compartment 200 may include a nonmetallic inorganic material. Examples of the nonmetallic inorganic material may include ceramic, quartz, or the like. In an implementation, the flow compartment 200 may include a material having a high thermal conductivity.

The second space S2 of the flow compartment 200 may be separate or isolated from the first space S1 of the chamber. In an implementation, a flow of a gas in the second space S2 may be blocked from entering or otherwise interacting with the first space S1. In an implementation, through-holes (for allowing a gas flow) may be formed in a lower portion of the sidewall 204 of the flow compartment 200 to communicate or open the second space S2 with the first space S1, so that the chamber and the flow compartment may have a same pressure.

The substrate support may include a plurality of support pins 300 supporting the substrate. The support pins 300 may extend upwardly from the lower wall 202 of the flow compartment 200 to contact and support the wafer W. Accordingly, the wafer W may be supported within the flow compartment 200 by the substrate support, and the heater may heat the wafer W to a desired temperature.

The gas pipe may include a plurality of carrier gas pipes for introducing and draining a carrier gas into and from the flow compartment 200. For example, the gas pipe may include a carrier gas inlet pipe 152 and a carrier gas outlet pipe 154 in, e.g., both, sidewalls 204 of the flow compartment 200 opposite to each other. For example, the carrier gas inlet pipe 152 may be arranged opposite to the carrier gas outlet pipe 154 in the flow compartment 200.

In an implementation, the thermal desorption system 100 may further include a gas supplier 150 to supply the carrier gas (e.g., nitrogen (N₂) gas) of or at a high temperature through the carrier gas inlet pipe 152. In an implementation, the thermal desorption system 100 may further include an analyzer 160 to detect a gas discharged through the carrier gas outlet pipe 162 to analyze a material absorbed on the surface of the substrate. For example, the analyzer 160 may analyze the discharged gas to determine the presence of material that had been absorbed on the surface of the substrate (and now removed by the carrier gas).

The gas supplier 150 may be connected to the carrier gas inlet pipe 152 through a first valve 153. The gas supplier 150 may supply the carrier gas into the flow compartment 200 within the chamber through the carrier gas inlet pipe 152. In an implementation, a mass flow controller (MFC) may be installed in the carrier gas inlet pipe 152 to control a flow of the carrier gas. In an implementation, the gas supplier 150 may supply the carrier gas into the first space S1 of the chamber and the second space S2 of the flow compartment 200 through an extra carrier gas inlet pipe.

The flow compartment 200 may provide the second space S2 as the separate gas flow space within the chamber space S1. The carrier gas introduced into the flow compartment 200 through the carrier gas inlet pipe 152 may flow through the space S2 over the substrate within the flow compartment 200, and then, may be discharged with a material thermally desorbed from the surface of the substrate into the analyzer 160 through the carrier gas outlet pipe 162.

The analyzer 160 may include at least one of a first analyzer 161A for performing quantitative analysis and a second analyzer 161B for performing qualitative analysis. As illustrated in FIG. 4, the analyzer 160 may include both the first analyzer 161A and the second analyzer 161B. The carrier gas outlet pipe 162 may be connected to a first outlet line 164 and a second outlet line 166 through a first control valve 163. The first analyzer 161A may be connected to the first outlet line 164, and the second analyzer 161B may be connected to the second outlet line 166. A second control valve 165 may be installed in the first outlet line 164, and a third control valve 167 may be installed in the second outlet line 166. A flow of the gas into the first and second analyzers 161A and 161B may be controlled by the first to third control valves 163, 165, and 167.

The first analyzer 161A may be an analyzer using, e.g., atmospheric pressure ionization (API) mass spectrometry, integrated cavity output spectroscopy (ICOS, for example, OA-ICOS), etc. The second analyzer 161B may be, e.g., a residual gas analyzer (RGA).

In an implementation, the thermal desorption system 100 may further include an exhaust to reduce a pressure of the chamber. The exhaust may include, e.g., an exhaust line 172 and a vacuum pump 170 connected to the exhaust line 172. The exhaust line 172 may be connected to the carrier gas inlet pipe 152 through first valve 153. The vacuum pump 170 may selectively create a vacuum within the first space S1 of the chamber and the second space S2 within the flow compartment 200 through the exhaust line 172 and the carrier gas inlet pipe 152.

In an implementation, the vacuum pump 170 may selectively create a vacuum within the first space S1 of the chamber and the second space S2 within the flow compartment 200 through an extra exhaust line.

The exhaust may include an electromagnetic valve installed in the exhaust line 172. A controller may open and close the electromagnetic valve, and operate the vacuum pump. Accordingly, the exhaust may discharge the gas within the chamber and the flow compartment 200 to the outside.

The thermal desorption system 100 may further include a temperature sensor to detect a temperature of the substrate or the chamber. For example, the temperature sensor may include a thermocouple 400 positioned within the flow compartment 200 to detect a temperature of the substrate. The thermocouple 400 may extend upwardly from the lower wall 202 of the flow compartment 200 to detect a temperature of the substrate. In an implementation, the temperature sensor may further include a second thermocouple positioned in the chamber and configured to a temperature of the chamber. For example, the second thermocouple may protrude from the upper surface 128 of the upper chamber 120 to detect a temperature of the upper chamber 120.

As illustrated in FIG. 5, the flow compartment 200 may be disposed on the bottom wall of the lower chamber 110. The upper wall 206 of the flow compartment 200 (e.g., a plane of a top surface of the upper wall 206) may be positioned to be lower than the upper edge 116 of the lower chamber 110 (e.g., a plane of the upper edge 116). An upper surface of the upper edge 116 of the lower chamber 110 may be positioned to be higher than an upper surface of the upper wall 206 of the flow compartment 200 by a predetermined height H.

As illustrated in FIG. 6, in an implementation, the upper wall 206 of the flow compartment 200 may be positioned to be higher than the upper edge 116 of the lower chamber 110. An upper surface of the upper wall 206 of the flow compartment 200 may be positioned to be higher than an upper surface of the upper edge 116 by a predetermined height. The upper surface of the upper wall 206 of the flow compartment 200 may be spaced apart from the upper surface 128 of the upper chamber 120 by a predetermined distance L.

As mentioned above, the thermal desorption system 100 may include the flow compartment 200 in the chamber to provide a separate gas flow space within the chamber, the heater to heat the wafer W loaded into the flow compartment 200 to desorb a material (e.g., a reaction by-product) on or from the surface of the wafer W, and the gas pipe to introduce the carrier gas into the flow compartment 200 and discharge the carrier gas with the desorbed material from the flow compartment 200. In an implementation, the thermal desorption system 100 may further include an analyzer 170 to analyze the discharged material in real time. In an implementation, the thermal desorption system 100 may perform the thermal desorption process and then may exhaust the gas (or air) within the chamber and the flow compartment 200 to clean the flow compartment 200.

The flow compartment 200 may provide a minimum gas flow space which is separate from the upper space of the chamber. A flow of the desorbed material within the flow compartment may be blocked to the upper space of the chamber, and the desorbed material may be discharged directly through the carrier gas outlet pipe 162 of the gas pipe and then may be analyzed in real time. Accordingly, the desorbed material may be prevented from being condensed due to a temperature difference between the wafer W and the upper space of the chamber, and a time for the adsorbed material to transfer from the wafer W to the analyzer 170 may be reduced to thereby improve efficiency of analysis.

In an implementation, the carrier gas may be introduced to the chamber, atmospheric pressure analysis may be performed, and the total amount of the material desorbed from the wafer W may be used to perform quantitative analysis in real time. Further, the thermal desorption system 100 may perform quantitative analysis and qualitative analysis optionally or at the same time.

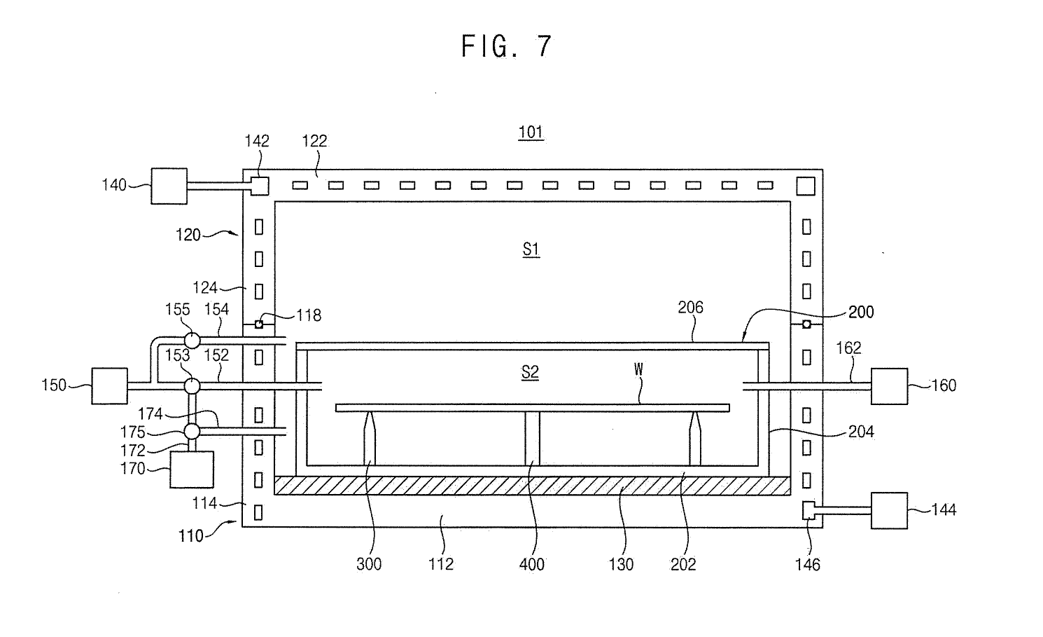

FIG. 7 illustrates a plan view of a thermal desorption system in accordance with example embodiments. The thermal desorption system may be substantially the same as or similar to the thermal desorption system as described with reference to FIG. 1, except for a gas pipe. Thus, same reference numerals will be used to refer to the same or like elements and any further repetitive explanation concerning the above elements may be omitted.

Referring to FIG. 7, a gas pipe of a thermal desorption system 101 may include a first carrier gas inlet pipe 152 to introduce a carrier gas into a second space S2 of a flow compartment 200 and a second carrier gas inlet pipe 154 to introduce a carrier gas into a first space S1 of a chamber.

A gas supplier 150 may supply the carrier gas into the flow compartment 200 through the first carrier gas inlet pipe 152. A first valve 153 may be installed in the first carrier gas inlet pipe 152 and a second valve 155 may be installed in the second carrier gas inlet pipe 154.

A vacuum pump 170 may exhaust an air within the flow compartment 200 through a first exhaust line 172 and the first carrier gas inlet pipe 152. Additionally, the vacuum pump 170 may exhaust an air within the chamber through the first exhaust line 172 and a second exhaust line 174.

In an implementation, the second space S2 of the flow compartment 200 may be sealed from the first space S1 of the chamber. Pressures in the first space S1 of the chamber and the second space S2 of the flow compartment 200 may be controlled independently to each other.

In an implementation, the second space S2 of the flow compartment 200 may be in communication with the first space S1 of the chamber. In this case, the gas supplier 150 may supply the carrier gas to the flow compartment 200 and the chamber respectively, and may exhaust the air within the flow compartment 200 and the chamber respectively.

Hereinafter, a method of analyzing a contamination material on a substrate using the thermal desorption system in FIG. 1 will be explained in detail.

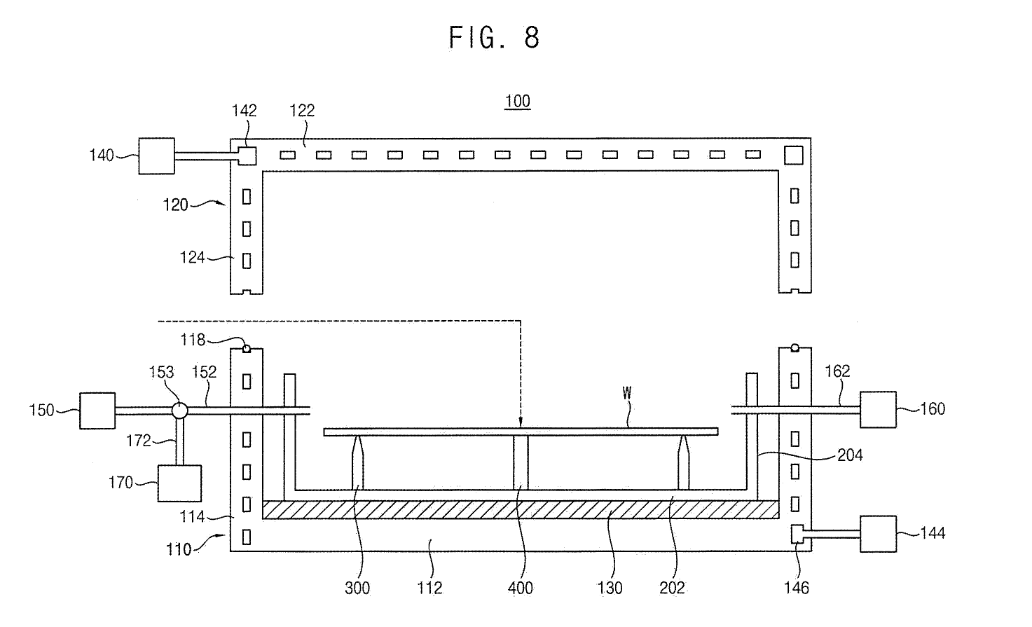

FIGS. 8 to 10 illustrate views of stages in a method of analyzing a substrate in accordance with example embodiments.

Referring to FIG. 8, first, a semiconductor process (e.g., a thin layer deposition process, an etch process, or the like) may be performed on a wafer W, and then, the wafer W may be loaded into a chamber of a thermal desorption system in order to analyze a contamination material on the wafer W.

An upper chamber 120 may move upwardly to open the chamber, and then, a flow compartment 200 within the chamber may be opened and the wafer W may be transferred onto supporting pins 300 of a substrate support within the flow compartment 200. Thus, the wafer W may be supported on a heater 130 by the substrate support.

After the wafer W is placed on the supporting pins 300, an upper wall 206 may be disposed on sidewalls 204 of the flow compartment 200, to form a space as a minimum gas flow path surrounding the wafer W within the chamber.

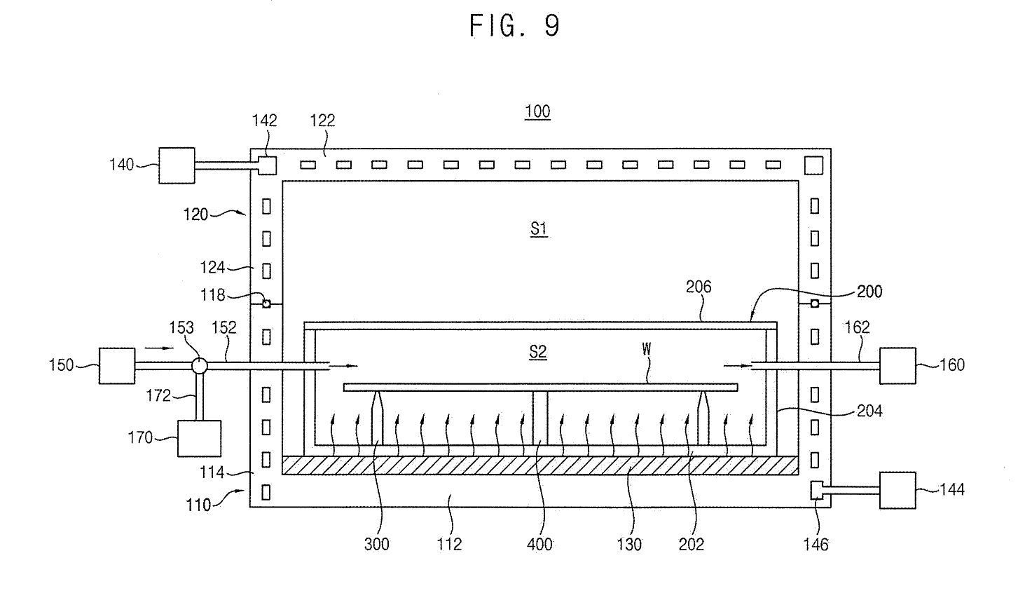

Referring to FIG. 9, the upper chamber 120 may move downwardly to engage with a lower chamber 110, a thermal desorption process may be performed by the heater that heats the wafer W to a desired temperature, and a carrier gas may be introduced into the flow compartment 200 and then may be exhausted from the flow compartment 200 to be analyzed in real time.

For example, first, the carrier gas may be supplied to the chamber to maintain the chamber under an atmospheric pressure. A gas supplier 150 may supply the carrier gas into the flow compartment 200 through a carrier gas inlet pipe 152. The carrier gas may include a nitrogen (N₂) gas. Accordingly, the chamber may be maintained under an atmospheric pressure. Before supplying the carrier gas into the chamber, a gas within the chamber may be exhausted to form a vacuum within the chamber.

Then, the wafer W may be heated to desorb (e.g., remove) a material from the wafer W. The heater 130 may heat the wafer W to a temperature of, e.g., about 600° C. to about 900° C. In an implementation, a coolant circulator may circulate a coolant through coolant lines 142 and 146 in the chamber to maintain the chamber under room or ambient temperature (e.g., under about 30° C.). In an implementation, the wafer W may be heated to about 900° C., and a temperature of an upper space of the chamber may be heated to about 200° C. For example, a relatively great temperature difference between the wafer W and the upper space of the chamber may be generated.

When the wafer W is heated, a carrier gas may be introduced into the flow compartment 200 and may be exhausted with a gas including the material desorbed from the wafer W from the flow compartment 200.

The gas supplier 150 may supply the carrier gas into the flow compartment 200 through the carrier gas inlet pipe 152. The carrier gas may include a nitrogen (N₂) gas of high temperature. The carrier gas may flow through the gas flow space within the flow compartment 200 and then may be exhausted with the gas including the material thermally desorbed from the wafer W from the flow compartment 200 through a carrier gas outlet pipe 162. Accordingly, the desorbed gas may not move upward to the upper space of the chamber having a relatively low temperature, but rather may be exhausted through the carrier gas outlet pipe 162 to an analyzer 160.

The discharged gas may be analyzed in real time. The analyzer 160 may analyze the discharged gas through the carrier gas outlet pipe 162. For example, the analyzer 160 may include at least one of a first analyzer 161A performing quantitative analysis and a second analyzer 161B performing qualitative analysis. Accordingly, the total amount of the material desorbed from the wafer W may be used to perform quantitative analysis in real time. Additionally, quantitative analysis and qualitative analysis may be performed optionally or at the same time.

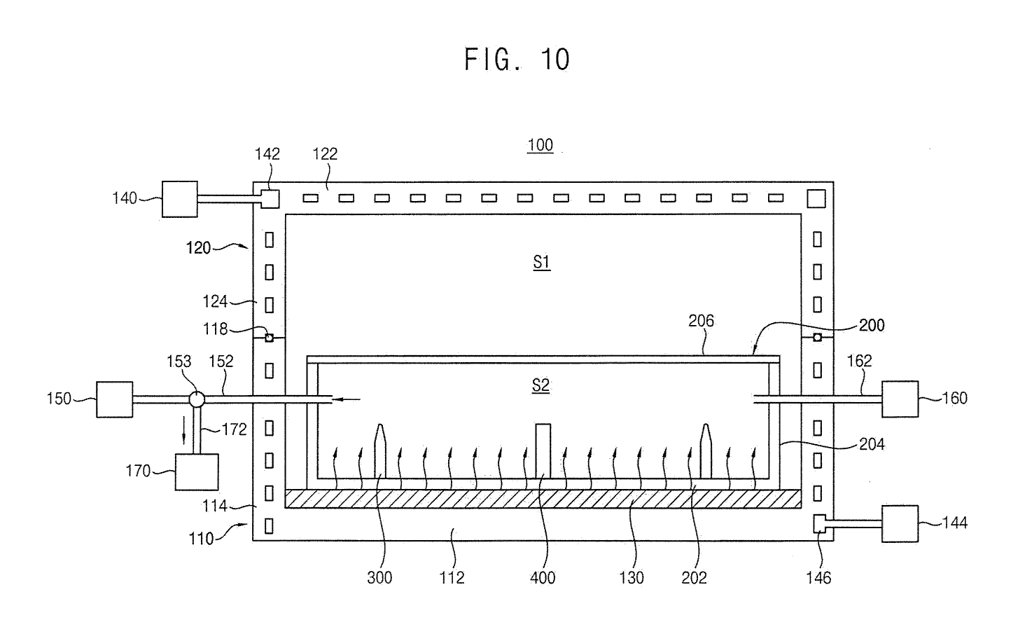

Referring to FIG. 10, after the wafer is unloaded, a gas within the chamber and the flow compartment 200 may be exhausted.

In an implementation, after analysis of the wafer W, the wafer W may be unloaded and then, a cleaning process of the chamber may be performed.

The heater 130 may heat the flow compartment 200 and the chamber to a desired temperature to desorb a material adsorbed within the flow compartment 200 and the chamber, and a vacuum pump 170 may exhaust the gas within the flow compartment 200 an the chamber through the first carrier gas inlet pipe 152. Thus, a remaining material within the flow compartment 200 may be removed completely, thereby improving efficiency of following or subsequent analyses.

The above thermal desorption system and the substrate analyzing method may be used to manufacture a semiconductor device such as a logic device or a memory device. For example, the semiconductor device may include logic devices such as central processing units (CPUs), main processing units (MPUs), application processors (APs), etc., volatile memory devices such as DRAM devices, SRAM devices, etc., or non-volatile memory devices such as flash memory devices, PRAM devices, MRAM devices, RRAM devices, etc.

By way of summation and review, a vacuum-based analysis method may be adapted in a thermal desorption system, it may be impossible to perform quantitative analysis, and it may be difficult to analyze a total amount of desorbed gas. Further, the desorbed gas may be adsorbed again or condensed within a chamber, and it may be difficult to precisely analyze the material adsorbed on the wafer.

The embodiments may provide a thermal desorption system for analyzing a material adsorbed on a wafer surface.

The flow compartment may provide a minimum gas flow space which is separate from an upper space of the chamber. The desorbed material may be discharged directly through a carrier gas outlet pipe connected to the flow compartment and then may be analyzed in real time. Accordingly, the desorbed material may be prevented from being condensed due to a temperature difference between the substrate and the upper space of the chamber, and a time for the adsorbed material to transfer from the substrate to the analyzer may be reduced to thereby improve efficiency of analysis.

Additionally, the total amount of the material desorbed from the substrate may be used to perform quantitative analysis in real time. Further, quantitative analysis and qualitative analysis may be performed optionally or at the same time.

The embodiments may provide a thermal desorption system capable of improving efficiency of analysis.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A thermal desorption system, comprising:
a chamber including a space in which a substrate is heated;
a flow compartment within the chamber, the flow compartment providing a separate gas flow space within the chamber;
a substrate support that supports the substrate within the flow compartment;
a heater that heats the substrate within the flow compartment; and
a gas pipe that introduces a carrier gas into the flow compartment and discharges the carrier gas from the flow compartment.

2. The thermal desorption system as claimed in claim 1, wherein the flow compartment is spaced apart from an inner upper surface of the chamber by a predetermined distance.

3. The thermal desorption system as claimed in claim 1, wherein the gas pipe includes a carrier gas inlet pipe and a carrier gas outlet pipe in sidewalls of the flow compartment, the carrier gas inlet pipe being arranged opposite to the carrier gas outlet pipe.

4. The thermal desorption system as claimed in claim 3, further comprising an analyzer that detects a gas discharged through the carrier gas outlet pipe and analyzes the discharged gas for the presence of a material absorbed on a surface of the substrate.

5. The thermal desorption system as claimed in claim 1, wherein the flow compartment includes:
a lower wall on a bottom wall of the chamber;
a plurality of sidewalls extending in a vertical direction on the lower wall; and
an upper wall on the sidewalls.

6. The thermal desorption system as claimed in claim 5, wherein the upper wall is movable to selectively cover the sidewalls such that the upper wall selectively opens and closes the flow compartment.

7. The thermal desorption system as claimed in claim 1, wherein the flow compartment includes a nonmetallic inorganic material.

8. The thermal desorption system as claimed in claim 1, wherein the substrate support includes a plurality of supporting pins on a lower wall of the flow compartment.

9. The thermal desorption system as claimed in claim 1, further comprising an exhaust that reduces a pressure of the chamber.

10. The thermal desorption system as claimed in claim 1, further comprising a coolant circulator that circulates a coolant through a coolant line in the chamber.

11. A thermal desorption system, comprising:
a chamber including a lower chamber and an upper chamber, the lower chamber and the upper chamber being engaged with each other to provide a first space;
a flow compartment within the chamber to provide a separate second space within the first space;
a substrate support that supports the substrate within the flow compartment;
a heater that heats the substrate within the flow compartment; and
a gas pipe that introduces and discharges a carrier gas into and from the flow compartment.

12. The thermal desorption system as claimed in claim 11, wherein the flow compartment is on a bottom wall of the lower chamber.

13. The thermal desorption system as claimed in claim 11, wherein the gas pipe includes a carrier gas inlet pipe and a carrier gas outlet pipe in sidewalls of the flow compartment, the carrier gas inlet pipe being arranged opposite to the carrier gas outlet pipe.

14. The thermal desorption system as claimed in claim 13, further comprising an analyzer that detects a gas discharged through the carrier gas outlet pipe and analyzes the discharged gas for the presence of a material absorbed on a surface of the substrate.

15. The thermal desorption system as claimed in claim 11, further comprising an exhaust that reduces a pressure of the chamber.

16. A thermal desorption system, comprising:
a flow compartment;
a substrate support in the flow compartment, a substrate being supportable on the substrate support;
a heater in the flow compartment, the substrate being heatable by the heater such that a material is desorbable from a surface of the substrate;
a gas pipe in fluid communication with the flow compartment, a carrier gas being introducible into the flow compartment and dischargeable from the flow compartment through the gas pipe; and
an analyzer that analyzes gas discharged through the gas pipe for the presence of a material desorbed from a surface of the substrate.

17. The thermal desorption system as claimed in claim 16, wherein the analyzer performs quantitative analysis or qualitative analysis of the discharged gas.

18. The thermal desorption system as claimed in claim 16, wherein the analyzer analyzes the discharged gas in real time.

19. The thermal desorption system as claimed in claim 16, wherein conditions in the flow compartment are maintained by the heater and the gas pipe such that material desorbed from the surface of the substrate is not condensed or reabsorbed on the substrate.

20. The thermal desorption system as claimed in claim 16, wherein the gas pipe includes a carrier gas inlet pipe in one sidewall of the flow compartment and a carrier gas outlet pipe in another sidewall of the flow compartment, the one sidewall being opposite to the other sidewall.
